(19) United States
(12) Patent Application Publication
(10) Pub. No.: US 2025/0098708 A1
(43) Pub. Date: Mar. 27, 2025

(54) TWO-STEP PRODUCTION METHOD OF MYCOPROTEIN BIOLOGICAL FEED BY FERMENTING CORN HUSKS

(71) Applicant: Inner Mongolia University of Technology, Hohhot (CN)

(72) Inventors: Zhanying LIU, Hohhot (CN); Yaping YAN, Hohhot (CN)

(21) Appl. No.: 18/936,264

(22) Filed: Nov. 4, 2024

Related U.S. Application Data
(63) Continuation of application No. PCT/CN2023/131435, filed on Nov. 14, 2023.

(30) Foreign Application Priority Data
Dec. 19, 2022 (CN) 202211628999.5

(51) Int. Cl. A23K 10/37; A23K 10/12; A23K 10/18; A23K 20/147; C12N 1/18; C12R 1/685; C12R 1/72; C12R 1/865

(52) U.S. Cl. CPC A23K 10/37; A23K 10/12; A23K 10/18; A23K 20/147; C12N 1/185; C12R 2001/685; C12R 2001/72; C12R 2001/865

(57) ABSTRACT

The present invention belongs to the technical field of biological fermented feeds, in particular to a two-step production method of a mycoprotein biological feed by fermenting corn husks. The present invention provides a preparation method for a mycoprotein biological feed, which comprises: inoculating Aspergillus niger microbial agent on a first fermentation substrate for first fermentation, to obtain a yeast seed; and inoculating a compound microbial agent in a second fermentation substrate to perform second fermentation. The compositions of the first fermentation substrate, the second fermentation substrate and the compound microbial agent are defined. The degradation of cellulose and hemicellulose in the corn husk raw material can be maximized and the protein content is increased through two fermentations, while taking into account the cost. A preparation method of the high-quality protein feed is provided for the feed industry.

The present application claims priority to the Chinese Patent Application with an application number of CN202211628999.5 and a title of “a two-step production method of a mycoprotein biological feed by fermenting corn husks”, filed to China National Intellectual Property Administration on Dec. 19, 2022, the disclosures of which are hereby incorporated by reference.

### TECHNICAL FIELD

The present invention belongs to the technical field of preparation of biological fermented feeds, in particular to a two-step production method of a mycoprotein biological feed by fermenting corn husks.

### BACKGROUND

With the development of animal husbandry and breeding industry, the shortage phenomenon of protein feed resources in China has become more and more serious, and a large number of protein feeds such as soybean meal and fish meal need to be imported from abroad every year, which leads to substantial increase in feed cost and affects food safety. Therefore, the feed industry is in urgent need of an unconventional protein feed to replace a conventional protein feed. At present, in the field of protein feed replacement, cheap industrial and agricultural by-products are generally used as raw materials, indigestible substances in the raw materials are degraded through fermentation of several microorganisms, and the protein content is increased.

Corn husk is one of the biggest by-products of fermentation enterprises that use corn as the raw material. At present, the most common treatment way is to sell the corn husks as a ruminant feed after washing and drying, which has the disadvantages of low nutritional value, poor palatability and poor feeding effect. In order to solve this problem, researchers have carried out related research on microbial fermentation of corn husks. For example, the Chinese patent with a publication number of CN112868906A discloses “a preparation method for dietary fiber feed of corn husks”, including the preparation of a composite microecological preparation, a composite enzyme preparation and a fermentation substrate. The invention converts corn husks, which are not easily digested and absorbed by animals, into high-quality feed raw material products containing nutrients such as protein, prebiotics, soluble dietary fibers and insoluble dietary fibers through bacteria- and enzyme synergy, and simultaneously increases the added value of corn husks. The Chinese patent with a publication number of CN110934223A discloses a process of preparing a protein feed through solid microbial fermentation of corn husks, which includes: mixing wet corn husks, wheat brans, shell powder, calcium phosphate and water, stirring, sterilizing, and naturally cooling to room temperature to obtain base fluid; mixing a Candida tropicalis seed solution and a Bacillus natto seed solution to obtain a mixed seed solution, inoculating the mixed seed solution into the base fluid, performing fermentation and cultivation for 24 h, then inoculating with a Streptococcus thermophilus seed solution and continuing to perform fermentation and cultivation for 48-60 h; stopping fermentation and drying the fermentation products to constant weight at 60-70° C. to obtain the protein feed. The invention solves the problems of high energy consumption and high pollution in the drying process of the corn husks in the prior art, and puts forward a process of preparing a protein feed through solid microbial fermentation of corn husks.

Although relevant research on microbial fermentation of corn husks has achieved certain results, there are still some problems such as high cost, poor strain safety, high fiber content in feed, low protein content, etc. Moreover, Candida tropicalis and Bacillus natto in the above invention patent are not in the “feed additive categories”, and subsequent production cannot be carried out.

### SUMMARY

The purpose of the present invention is to provide a two-step production method of a mycoprotein biological feed by fermenting corn husks. By adopting the preparation method of the present invention, the fiber in the fermented feed can be degraded to the maximum while taking into account the cost, and the protein content of the fermented feed can be increased, so as to provide a preparation method for a high-quality protein feed for the feed industry, and the strain used is safe.

The present invention provides a preparation method for a mycoprotein biological feed, which comprises the following steps:
inoculating Aspergillus niger microbial agent on a first fermentation substrate for first fermentation, to obtain a yeast seed;
inoculating a compound microbial agent in a second fermentation substrate to perform second fermentation to obtain a mycoprotein biological feed;
The first fermentation substrate comprises the following raw materials by mass parts: 84-92 parts of corn husks, 4-6 parts of brans, 2-4 parts of soybean meal and 2-5 parts of wheat middlings;
The second fermentation substrate comprises the following raw materials by mass parts: 19.5-30 parts of corn husks, 3-6 parts of molasses, 2-4 parts of ammonium sulfate, 0.1-0.2 part of potassium dihydrogen phosphate, 0.1-0.2 part of magnesium sulfate, 0.10-0.20 part of sodium chloride, 0.10-0.15 part of acetic acid and 10-35 parts of the yeast seed;
The compound microbial agent comprises Saccharomyces cerevisiae and Candida utilis.

Preferably, the inoculation amount of the Aspergillus niger is 5-10% of the mass of the first fermentation substrate;

The effective viable count of the Aspergillus niger is 1.0×10⁹-2.0×10⁹ CFU/mL.

Preferably, the Aspergillus niger comprises Aspergillus niger CJH-JXSFZh-B703 with a collection number of CGMCC No. 22439.

Preferably, the time of the first fermentation is 24-48 h, and the temperature is 30-35° C.

Preferably, the inoculation amount of the compound microbial agent is 5%-10% of mass of the corn husks in the second fermentation substrate;

The effective viable count of the Saccharomyces cerevisiae is independently 1.5×10⁸ CFU/mL;

The effective viable count of the Candida utilis is independently 1.8×10⁸ CFU/mL.

A ratio of the effective viable counts of the Saccharomyces cerevisiae and the Candida utilis in the compound microbial agent is 1.5-10.5:1.8-12.6.

Preferably, the Saccharomyces cerevisiae comprises Saccharomyces cerevisiae CICC 32236; and the Candida utilis comprises Candida utilis CGMCC 2.2878.

Preferably, the fermentation time is 48-72 h and the temperature is 30-32° C.

The present invention provides a mycoprotein biological feed, which is prepared by the preparation method described in the technical solution.

Preferably, the effective viable count of the Saccharomyces cerevisiae in the mycoprotein biological feed is 1.4×10⁹ CFU/kg-1.4×10¹¹ CFU/kg, and the effective viable count of the Candida utilis is preferably 1.9×10⁹ CFU/kg-1.9×10¹¹ CFU/kg.

The present invention provides an application of the mycoprotein biological feed of the above technical solution in preparation of an animal feed.

The present invention provides an application of the mycoprotein biological feed of the above technical solution in improvement of milk yield and milk quality of animals.

### Beneficial Effects

The present invention provides the preparation method of the mycoprotein biological feed, comprising: inoculating Aspergillus niger microbial agent on a first fermentation substrate for first fermentation, to obtain a yeast seed; and inoculating a compound microbial agent in a second fermentation substrate to perform fermentation to obtain a mycoprotein biological feed. In addition, the compositions of the first fermentation substrate, the second fermentation substrate and the compound microbial agent are defined. The fermentation substrates that mainly comprise corn husks are treated by step fermentation, which can maximize the degradation of fiber in the fermented feed and increase the protein content while taking into account the cost. The strains adopted are highly safe, thereby providing a preparation method of the high-quality protein feed for the feed industry. The obtained mycoprotein biological feed can not only provide the high-quality protein, but also provide a variety of probiotics and enzymes, which has certain effects of reducing or eliminating the use of antibiotics and ensuring the green development of the breeding industry while ensuring the supply of the protein, and has wide application prospects. Moreover, the by-product after corn starch processing is processed for a second time in the present invention, which not only provides a high-quality and low-price antibiotic-free mycoprotein microbial feed for animal husbandry and breeding industry, but also solves the problem of inadequate utilization of corn husks as the current by-product of corn processing and improves the added value. The used solid-state fermentation mode has the characteristics of simple equipment structure, less investment, low energy consumption and simple operation. All fermentation products are products, have no other waste and less pollution.

### DESCRIPTION OF DRAWINGS

In order to more clearly describe the technical solution in the embodiment of the present invention or the prior art, the drawings required for use in the embodiment will be briefly introduced below.

FIG. 1 is a diagram of a mycelium state in the first fermentation product;

FIG. 2 is an experimental flow chart of embodiment 1, wherein the second fermentation substrate in FIG. 2 refers to dried corn husks, molasses, ammonium sulfate, potassium dihydrogen phosphate, sodium chloride and magnesium sulfate in the second fermentation substrate.

### MICROBIAL PRESERVATION INFORMATION

Aspergillus niger CJH-JWSFZh-B703, classified and named as Aspergillus niger, was collected in the China General Microbiological Culture Collection Center (CGMCC) on Jun. 8, 2021, at Institute of Microbiology, Chinese Academy of Sciences, No. 2, No. 1 Yard, Beichen West Road, Chaoyang District, Beijing, China. The postal code is 100101 and the collection number is CGMCC No. 22439.

### DETAILED DESCRIPTION

If there is no special requirement, the raw materials used in the present invention are routinely purchased by those skilled in the art.

The present invention provides a preparation method for a mycoprotein biological feed, which comprises the following steps:
inoculating Aspergillus niger microbial agent on a first fermentation substrate for first fermentation, to obtain a yeast seed;
inoculating a compound microbial agent in a second fermentation substrate to perform second fermentation to obtain a mycoprotein biological feed;
The first fermentation substrate comprises the following raw materials by mass parts: 84-92 parts of corn husks, 4-6 parts of brans, 2-4 parts of soybean meal and 2-5 parts of wheat middlings;
The second fermentation substrate comprises the following raw materials by mass parts: 19.5-30 parts of corn husks, 3-6 parts of molasses, 2-4 parts of ammonium sulfate, 0.1-0.2 part of potassium dihydrogen phosphate, 0.1-0.2 part of magnesium sulfate, 0.10-0.20 part of sodium chloride, 0.10-0.15 part of acetic acid and 10-35 parts of the yeast seed;
The compound microbial agent comprises Saccharomyces cerevisiae and Candida utilis.

The present invention inoculates the Aspergillus niger microbial agent on the first fermentation substrate for first fermentation, to obtain the yeast seed. The Aspergillus niger in the present invention is preferably Aspergillus niger CJH-JXSFZh-B703. The collection number of the Aspergillus niger CJH-JXSFZh-B703 is CGMCC No. 22439. The number of spores of the Aspergillus niger in the the yeast seed is preferably 6.52×10⁹ CFU/kg. The Aspergillus niger used in the present invention is a safe strain permitted as a microbial feed additive in the “feed additive categories”. Moreover, enzymes produced by Aspergillus niger are abundant, and more than ten kinds of enzymes that can be used as feed enzymes permitted by the Ministry of Agriculture can be derived from Aspergillus niger, wherein cellulase, protease, amylase and pectinase can act on corn husks. Moreover, the Aspergillus niger contains enzymes, and the highest activity of the enzymes is 39.52 U/mL. Aspergillus niger has a strong cellulose degradation ability. If Aspergillus niger is inoculated into corn husks, Aspergillus niger can grow well through some nutrients in corn husks and degrades cellulose into glucose and other small molecular substances for bacteria to grow. The treated corn husks are rich in mycoprotein, amino acids, vitamins, minerals and other elements, and are used as the feed which has deep utilization value.

Before the present invention inoculates Aspergillus niger on the first fermentation substrate for the first fermentation, Aspergillus niger is preferably subjected to slant culture in a malt agar medium. After the slant culture, the Aspergillus niger obtained from the slant culture is selected and inoculated into a malt extract liquid medium for shake-flask culture to obtain an Aspergillus niger microbial agent.

In the present invention, the temperature of the slant culture is preferably 30° C. During the slant culture, the inoculation amount of Aspergillus niger is preferably 1 loop. The preparation method of the malt agar medium in the present invention preferably comprises the following steps: mixing malt extract, agar and water to obtain a malt agar medium, wherein the dosage of the malt extract is preferably 1.0 L; the dosage of the agar is preferably 15.0 g; the dosage of the water is preferably the dosage when the Brix of the malt extract is adjusted to 12° Brix; the malt agar medium is preferably a natural pH; and the malt agar medium is preferably a malt agar medium obtained by sterilization at 121° C. for 20 min.

The inoculation amount of the shake-flask culture in the present invention is preferably 1 loop. The temperature of the shake-flask culture is preferably constant temperature of 30° C. The rotating speed of the shake-flask culture is preferably 180 r/min. The time of the shake-flask culture is preferably 24 h. In the present invention, the malt extract liquid medium is preferably a maltose solution with an apparent Brix of 12° Brix, and the malt extract liquid medium is preferably a malt extract liquid medium obtained by sterilization at 115° C. for 30 min. The spore number of the Aspergillus niger microbial agent in the present invention is preferably ≥10⁹ CFU/mL, more preferably 1.0×10⁹-2.0×10⁹ CFU/mL, most preferably 1.0×10⁹ CFU/mL.

After the Aspergillus niger microbial agent is obtained, the present invention preferably adjusts the Aspergillus niger microbial agent so that the spore number is 1.0×10⁹-2.0×10⁹ CFU/mL, and then inoculates the Aspergillus niger microbial agent on the first fermentation substrate. The mode of regulation is not limited in the present invention, and the mode known to those skilled in the art can be adopted. Preferably, sampling is performed every 2 h; the spore number of Aspergillus niger in the Aspergillus niger microbial agent is monitored in real time; and the spore number of Aspergillus niger is calculated through a blood cell counter. The sampling preferably refers to taking a seed solution of Aspergillus niger.

In the present invention, the first fermentation substrate comprises the following raw materials by mass parts: 84-92 parts of corn husks, 4-6 parts of brans, 2-4 parts of soybean meal and 2-4 parts of wheat middlings.

Based on mass parts, the first fermentation substrate of the present invention comprises 84-92 parts of corn husks, preferably comprises 86-90 parts and more preferably comprises 86 parts. The corn husks of the present invention preferably comprise dry corn husks, more preferably dry corn husks. The type of the corn husks preferably comprises coarse corn husks and/or fine corn husks, and more preferably comprises coarse corn husks and fine corn husks. The mass ratio of the coarse corn husks and the fine corn husks is preferably 66-82:10-20, and more preferably 66:20, 72:10 or 80:10. The particle size of the coarse corn husks is preferably 5 meshes to 10 meshes, and the fine corn husks are preferably corn husks that have a passing rate of more than 85% under a 20-mesh sieve. That is, the fine corn husks are preferably the screened substances obtained after the corn husks are crushed and screened through the 20-mesh sieve, but there may be fine corn husks with a particle size slightly larger than 20 meshes in the crushing process. The purpose of selecting corn husks with different meshes is to control the gap between the corn husks, to avoid excessive gaps caused by excessive coarse corn husks or excessive fine corn husks, which is not easy to control the water content, or makes the corn husks too thick. The water content of the dry corn husks in the present invention is preferably 9 wt. %. The present invention has no special limitation on the source of the corn husks, which can be obtained by routine purchase by those skilled in the art. The corn husks in the specific embodiment of the present invention are purchased from Inner Mongolia Fufeng Biotechnology Co., Ltd. The water content of the first fermentation substrate of the present invention is preferably controlled as 35 wt. %-40 wt. %.

Based on the mass part of the corn husks, the first fermentation substrate of the present invention comprises 4-6 parts of brans, preferably comprises 5-6 parts, and most preferably comprises 6 parts.

Based on the mass part of the corn husks, the first fermentation substrate of the present invention comprises 2-4 parts of soybean meal, preferably comprises 2.5-4 parts, and most preferably comprises 4 parts.

Based on the mass part of the corn husks, the first fermentation substrate of the present invention comprises 2-4 parts of wheat middlings, preferably comprises 2.5-5 parts, and most preferably comprises 4-5 parts.

Before inoculation, the present invention preferably carries out the first steaming, the first stirring, water addition, the second stirring, the second steaming and the third stirring on the first fermentation substrate to obtain steaming material. The temperature of the first steaming and the second steaming is preferably 115° C.-120° C., more preferably 120° C. The air pressure of the first steaming and the second steaming is preferably 0.1 MPa-0.2 MPa, more preferably 0.2 MPa. The time of the first stirring and the second stirring is preferably 10 min; and the time of the third stirring is preferably 20 min. When the present invention adds water, the use amount of water is 60%-80% of the mass of the first fermentation substrate, more preferably 80%. Steaming under the conditions set by the present invention can eliminate other harmful hybrid bacteria in the first fermentation substrate, and can also expand the fiber structure and promote hydrolysis. The water content of the steaming material of the present invention is preferably 40 wt. %-60 wt. %, further preferably 43 wt. %-58 wt. %, and more preferably 45 wt. %-55 wt. %. The initial pH value of the steaming material is preferably 7.0±0.2, that is, the pH value of the steaming material is 7.0±0.2.

When the Aspergillus niger is inoculated in the present invention, the inoculation amount of the Aspergillus niger is preferably 5%-10% of the mass of the steaming material, and more preferably 5%.

After inoculation with the Aspergillus niger microbial agent, the present invention carries out expanded inoculation and first fermentation on the steaming material inoculated with the Aspergillus niger microbial agent, to obtain a yeast seed, also known as a first fermentation product. The temperature of the expanded inoculation in the present invention is preferably 30° C.; and the time of the expanded inoculation is preferably 48 h. In the present invention, the time of the first fermentation is preferably 24-48 h, further preferably 28-45 h, more preferably 30-42 h, and most preferably 33-37 h. The indoor temperature of the first fermentation is preferably 25° C.-30° C., that is, during the first fermentation, the temperature in the environment is preferably 25° C.-30° C.; during the first fermentation, the temperature of the fermentation product is preferably 30-35° C.; the relative humidity of the first fermentation is preferably ≥95° C.; and the temperature of the fermentation product of the present invention preferably refers to the temperature of the steaming material inoculated with the Aspergillus niger microbial agent during the first fermentation. When the temperature of the fermentation product exceeds 35° C. and cannot be reduced to be less than 35° C. through continuous ventilation, the present invention preferably conducts cooling by turning or stirring. The present invention has no limitation on the mode of turning and stirring, and the mode known to those skilled in the art may be used.

After the yeast seed is obtained, the present invention inoculates the compound microbial agent on the second fermentation substrate to carry out the second fermentation to obtain a mycoprotein biological feed. In the present invention, the compound microbial agent comprises Saccharomyces cerevisiae and Candida utilis. The effective viable count of the Saccharomyces cerevisiae is independently 1.5×10⁸ CFU/mL. The effective viable count of the Candida utilis is independently 1.8×10⁸ CFU/mL. A ratio of the effective viable counts of the Saccharomyces cerevisiae and the Candida utilis in the compound microbial agent is 1.5-10.5:1.8-12.6. The present invention preferably mixes the Saccharomyces cerevisiae and the Candida utilis by a volume ratio to obtain the compound microbial agent. The volume ratio of the Saccharomyces cerevisiae and the Candida utilis is preferably 1-7:1-7, and more preferably 1:1-2, 1-2:1, 1.5:1-2, 7:3 or 3:7.

In the present invention, the Saccharomyces cerevisiae is preferably Saccharomyces cerevisiae with collection number of CICC 32236, and the Saccharomyces cerevisiae CICC 32236 is a strain of China Center of Industrial Culture Collection (CICC). CICC has a special website at a website address of http://www.china-cicc.org. The public can directly order strains online. The website of the Saccharomyces cerevisiae CICC 32236 is http://www.china-cicc.org/search/?classtype=0&keyword=32236. The public can obtain the strain from CICC. The present invention has no special limitation on the source of the Saccharomyces cerevisiae, which can be obtained by routine purchase by those skilled in the art. In the specific embodiment of the present invention, the Saccharomyces cerevisiae is preferably purchased from the CICC.

The Candida utilis of the present invention is preferably Candida utilis with collection number of CGMCC 2.2878, and the Candida utilis CGMCC 2.2878 is a strain of the CGMCC. CGMCC has a special website at the website address of https://cgmcc.net/english/, and the public can directly order the strain online. The website of Candida utilis CGMCC 2.2878 is https://cgmcc.net/english/detail?id=66762, and the public can obtain the strain from CGMCC. The present invention has no special limitation on the source of the Candida utilis, which can be obtained by routine purchase by those skilled in the art. In the specific embodiment of the present invention, the Candida utilis is preferably purchased from CGMCC. The Saccharomyces cerevisiae used in the present invention is rich in nutrients such as protein, amino acid and small peptide, can improve the palatability and nutritional value of feed after fermentation by Saccharomyces cerevisiae, and increases animal feed intake and digestive utilization rate. The Candida utilis has a high protein content, accounting for 32%-75% of the dry weight, is rich in nutrients such as fat, vitamins and nucleic acids, has wide range of carbon source utilization, and can use both pentaglucose and hexose. Moreover, the true protein content is increased greatly after the two are fermented together.

The present invention further preferably provides a preparation method of a compound microbial agent, comprising the following steps:
inoculating Saccharomyces cerevisiae and Candida utilis on a YPD agar medium respectively for slant culture;
inoculating Saccharomyces cerevisiae and Candida utilis obtained from slant culture in a YPD liquid medium respectively for shaking culture to obtain the seed solution of Saccharomyces cerevisiae and the seed solution of the Candida utilis;
mixing the seed solution of Saccharomyces cerevisiae and the seed solution of the Candida utilis by a volume ratio of 1-7:1-7 to obtain the compound microbial agent.

The present invention preferably inoculates Saccharomyces cerevisiae and Candida utilis respectively on the YPD agar medium for slant culture. The temperature of the slant culture in the present invention is preferably 30° C. During the slant culture of Saccharomyces cerevisiae, a single colony of 1-loop Saccharomyces cerevisiae is preferably selected and plotted in the YPD agar medium. During the slant culture of Candida utilis, a single colony of 1-loop Candida utilis is preferably selected and plotted in the YPD agar medium. The YPD agar medium of the present invention preferably comprises the following components of mass: 10 g of peptone, 5 g of yeast extract powder, 10 g of glucose, 20 g of agar powder and 1 kg of distilled water. The pH of the YPD agar medium is preferably natural. The YPD agar medium is preferably a YPD agar medium obtained by sterilization at 115° C. for 30 min.

The present invention preferably inoculates Saccharomyces cerevisiae and Candida utilis obtained from slant culture respectively in a YPD liquid medium for shaking culture to obtain the seed solution of Saccharomyces cerevisiae and the seed solution of Candida utilis. The inoculation amount of the shaking culture of the present invention is preferably 1 loop. The temperature of the shake-flask culture is preferably constant temperature of 30° C. The rotating speed of the shake-flask culture is preferably 180 r/min. The time of the shake-flask culture is preferably 11 h. In the present invention, the YPD liquid medium preferably comprises the following components of mass: 10 g of peptone, 5 g of yeast extract powder, 10 g of glucose and 1 L of distilled water. The pH of the YPD liquid medium is 7.0-7.4. The YPD liquid medium is preferably a YPD liquid medium obtained after sterilization at 115° C. for 30 min.

After the seed solution of Saccharomyces cerevisiae and the seed solution of Candida utilis are obtained, the present invention also preferably adjusts the seed solution of Saccharomyces cerevisiae and the seed solution of Candida utilis respectively, so that the effective viable count of the seed solution of Saccharomyces cerevisiae is 1.5×10⁸ CFU/mL and the effective viable count of the seed solution of Candida utilis is 1.8×10⁸ CFU/mL. The mode of regulation is not limited in the present invention, and the mode known to those skilled in the art can be adopted. In the specific embodiment of the present invention, the mode of regulation is preferably sampling every 2 h; the viable count of Saccharomyces cerevisiae in the seed solution of Saccharomyces cerevisiae and the viable count of Candida utilis in the seed solution of Candida utilis are monitored in real time; and the viable count of Saccharomyces cerevisiae or the viable count of Candida utilis is calculated respectively through a blood cell counter. The sampling preferably refers to taking the seed solution of Saccharomyces cerevisiae or the seed solution of Candida utilis.

The second fermentation substrate of the present invention comprises the following raw materials by mass parts: 25-30 parts of corn husks, 3-6 parts of molasses, 2-4 parts of ammonium sulfate, 0.1-0.2 part of potassium dihydrogen phosphate, 0.1-0.2 part of magnesium sulfate, 0.10-0.15 part of sodium chloride, 0.10-0.15 part of acetic acid and 10-35 parts of the yeast seed. In the present invention, the water content of the second fermentation substrate is preferably controlled to 70 wt. %, so that the texture of the second fermentation substrate is slightly sticky.

Based on mass parts, the second fermentation substrate of the present invention comprises 25-30 parts of corn husks, and preferably comprises 26-28 parts. The corn husks of the present invention are dry corn husks. The type of the corn husks preferably comprises coarse corn husks and fine corn husks. The mass ratio of the coarse corn husks and the fine corn husks is preferably 66-82:10-20. The particle size of the coarse corn husks is preferably 5 meshes to 10 meshes, and the fine corn husks are preferably corn husks that have a passing rate of more than 85% under a 20-mesh sieve. That is, the fine corn husks are preferably the screened substances obtained after the corn husks are crushed and screened through the 20-mesh sieve, but there may be fine corn husks with a particle size slightly larger than 20 meshes in the crushing process. The water content of the corn husks in the present invention is preferably 9 wt. %. The present invention has no special limitation on the source of the corn husks, which can be obtained by routine purchase by those skilled in the art.

Based on the mass part of the corn husks, the second fermentation substrate of the present invention comprises 3-6 parts of molasses, and preferably comprises 3.2-3.8 parts. In the present invention, there is no special limitation on the source of the molasses, which can be obtained by routine purchase by those skilled in the art. In the specific embodiment of the present invention, the molasses are preferably purchased from Inner Mongolia Fufeng Biotechnology Co., Ltd.

Based on the mass part of the corn husks, the second fermentation substrate of the present invention comprises 2-4 parts of ammonium sulfate, and preferably comprises 2-3 parts.

Based on the mass part of the corn husks, the second fermentation substrate of the present invention comprises 0.1-0.2 part of potassium dihydrogen phosphate, and preferably comprises 0.12-0.19 part.

Based on the mass part of the corn husks, the second fermentation substrate of the present invention comprises 0.1-0.2 part of magnesium sulfate, and preferably comprises 0.13-0.18 part.

Based on the mass part of the corn husks, the second fermentation substrate of the present invention comprises 0.10-0.15 part of sodium chloride, and preferably comprises 0.1 part.

Based on the mass part of the corn husks, the second fermentation substrate of the present invention comprises 0.10-0.15 part of acetic acid, and preferably comprises 0.1 part.

Based on the mass part of the corn husks, the second fermentation substrate of the present invention comprises 10-35 parts of the yeast seed, preferably comprises 10-10.5 parts, and more preferably comprises 10.5 parts.

Before inoculation of the compound microbial agent, the present invention further preferably comprises: steaming the corn husks, the molasses, the ammonium sulfate, the potassium dihydrogen phosphate, the magnesium sulfate and the sodium chloride in the second fermentation substrate and adding water to obtain steaming material. The temperature of the steaming is preferably 115° C.-121° C., more preferably 121° C. The air pressure of the steaming is preferably 0.1 MPa-0.2 MPa, more preferably 0.2 MPa. The time of the steaming is 20 min. When the present invention adds water, the use amount of water is preferably 70% of the total mass of the water and the corn husks.

After the steaming material is obtained, the present invention further preferably comprises mixing the steaming material with the yeast seed and performing enzymatic hydrolysis, to obtain an enzymatic hydrolysis substrate. The time of the enzymatic hydrolysis is preferably 6 h, and the temperature of the enzymatic hydrolysis is preferably 25° C.-30° C. The present invention preferably adds tap water to the steaming material during enzymatic hydrolysis. The use amount of the tap water is preferably the amount of water when the steaming material becomes slightly sticky after adding the tap water.

After the enzymatic hydrolysis substrate is obtained, the present invention preferably mixes the enzymatic hydrolysis substrate with acetic acid, and inoculates the mixture obtained after the enzymatic hydrolysis substrate is mixed with acetic acid with the compound microbial agent for second fermentation. The time of the second fermentation of the present invention is preferably 48-72 h, more preferably 48 h. The temperature of the second fermentation is preferably 30-32° C. The present invention performs feeding in the second fermentation process. The specific steps of the feeding are as follows: at the 24 h of the second fermentation, adding 1% of ammonium sulfate and 1% of molasses before turning; at the 48 h of the second fermentation, adding 1% of ammonium sulfate and 1% of molasses before turning, wherein the use amounts of ammonium sulfate and molasses are preferably calculated by the weight of the second fermentation substrate. The present invention adopts the step feeding method to supplement the carbon source, trace elements and inorganic salt required for the growth and metabolism of the bacteria, so that the coarse fiber in the corn husks of the second fermentation substrate can be fully degraded, and the obtained components after degradation can contain higher nutrients after fermentation.

When the compound microbial agent is inoculated in the present invention, the inoculation amount of the compound microbial agent is preferably 5% of the mass of the corn husks in the second fermentation substrate.

The effective viable count of Saccharomyces cerevisiae in the mycoprotein biological feed of the present invention is preferably 1.4×10⁹ CFU/kg-1.4×10¹¹ CFU/kg, more preferably 1.4×10¹⁰ CFU/kg-1.4×10¹¹ CFU/kg, and most preferably 1.4×10¹¹ CFU/kg. The effective viable count of Candida utilis in the mycoprotein biological feed is preferably 1.9×10⁹ CFU/kg-1.9×10¹¹ CFU/kg, more preferably 1.9×10¹⁰ CFU/kg-1.9×10¹¹ CFU/kg, and most preferably 1.9×10¹¹ CFU/kg.

The present invention adopts Aspergillus niger to carry out the first fermentation for the first fermentation substrate to obtain a yeast seed, and then adopts a compound bacterial solution containing Saccharomyces cerevisiae and Candida utilis to carry out the fermentation treatment for the second fermentation substrate. It can be seen that the technology adopted by the present invention is multi-strain mixed step fermentation, and the cooperation among microorganisms greatly promotes the rapid degradation and fermentation of the corn husks, and has the following characteristics: 1. multi-strain symbiosis, rich in abundant cellulase, protease and amylase, and achieving enzyme complementation; 2. saving energy, simplifying technological equipment, and improving biochemical reaction; 3. abundant substrate, and capable of providing nutrients in stages; 4. nutrients such as monosaccharides generated by the enzymatic action are immediately utilized by other microorganisms, which eliminates the inhibitory effect of enzymatic hydrolysis products on enzymes while maintaining the concentration of degradants; 5. through the mutualism and commensalism relationship between the microorganisms, soybean meal can be hydrolyzed to produce small peptide substances, and the corn husks are fermented into biological fermented feed products that integrate the characteristics of nutrition, immunity, anti-infection and easy absorption. Moreover, the method of the present invention can maximize the degradation of the fiber in the fermented feed and increase the protein content while taking into account the cost.

The present invention further provides a mycoprotein biological feed prepared by the method of the above technical solution. The preparation method of the present invention has been discussed in detail above, and will not be repeated here. The raw material combination used by the mycoprotein biological feed provided by the present invention has low cost and wide source. The mycoprotein biological feed of the present invention can hydrolyze soybean meal in the raw material into small peptide substances, and cellulose and hemicellulose in the corn husks can be degraded into soluble sugar, and converted into mycoprotein. In addition, after the multi-strain fermentation, aromatic flavor is produced, which can also increase palatability. At the same time, the feed contains a large number of beneficial microorganisms and enzymes, wherein Aspergillus niger and Saccharomycetes are beneficial microorganisms in the rumen of dairy cows. Various cellulose and hemicellulose hydrolases produced can help the rumen to degrade crude fiber in the diet and improve the utilization rate of the feed. Moreover, the feed contains a variety of Saccharomycetes, abundant protein, B vitamins and amino acids, and can be widely used as a protein supplement for animal feed. The feed can promote the growth and development of animals, shorten the feeding period, and improve the yield and quality of products, such as milk yield and milk quality. Moreover, the feed contains Aspergillus niger, which can produce cellulase, hemicellulase and other hydrolases and glucoamylase, so as to improve the utilization rate of roughage and save feeding cost.

According to the above advantages, the present invention provides the mycoprotein biological feed in the above technical solution or the application of the mycoprotein biological feed in the above technical solution in the preparation of the animal feed. The animals of the present invention preferably comprise mammals, further preferably comprise cows, and more preferably comprise dairy cows.

According to the above advantages, the present invention provides the application of the specific protein feed in the above technical solution in improving the milk yield and milk quality of the animals. The mycoprotein biological feed of corn husks produced by the present invention contains a variety of Saccharomycetes, abundant protein, B vitamins and amino acids, wherein the protein content is about 30%-40%, and can be widely used as a protein supplement for animal feed. The feed can promote the growth and development of animals, shorten the feeding period, and improve the yield and quality of products, such as milk yield and milk quality.

The present invention further provides the application of Aspergillus niger, Saccharomyces cerevisiae and Candida utilis in the preparation of the mycoprotein biological feed. The Aspergillus niger, Saccharomyces cerevisiae and Candida utilis of the present invention have been described in detail above, and will not be repeated here.

To further explain the present invention, the technical solutions provided by the present invention are described in detail below in combination with the drawings and embodiments, but shall not be understood as the limitation of the protection scope of the present invention.

### Preparation Example

### Medium Preparation:

A malt agar medium is a malt agar medium of 12° Brix, specifically comprising 1.0 L of malt extract, 15.0 g of agar and the balance of water, and is sterilized at 121° C. for 20 min at natural pH.

A malt extract liquid medium is a maltose solution with an apparent Brix of 12°Brix.

A YPD agar medium comprises 10 g of peptone, 5 g of yeast extract powder, 10 g of glucose, 20 g of agar powder and 1 L of distilled water, has a pH of 7.0-7.4 and is sterilized at 15° C. for 30 min.

A YPD liquid medium comprises 10 g of peptone, 5 g of yeast extract powder, 10 g of glucose and 1 L of distilled water, has a pH of 7.0-7.4 and is sterilized at 15° C. for 30 min.

Aspergillus niger CJH-JXSFZh-B703 is subjected to slant culture in the malt agar medium. A single colony of 1-loop Aspergillus niger is selected and plotted in the malt agar medium and cultured at 30° C. After slant culture, 1-loop Aspergillus niger CJH-JXSFZh-B703 bevel is selected, inoculated into 100 mL of malt extract liquid medium contained in a 250 mL triangular bottle, and cultured at constant temperature of 30° C. and 180 r/min for 24 h to prepare a seed solution of Aspergillus niger CJH-JXSFZh-B703. The concentration of the seed solution is adjusted so that the effective viable count is 1.0×10⁹ CFU/mL, to obtain an Aspergillus niger microbial agent for use.

Saccharomyces cerevisiae CICC 32236 is subjected to slant culture in the YPD agar medium. A single colony of 1-loop Saccharomyces cerevisiae is selected and plotted in the YPD agar medium, and cultured at 30° C. After slant culture, 1-loop Saccharomyces cerevisiae CICC 32236 bevel is selected, inoculated into 100 mL of YPD liquid medium contained in a 250 mL triangular bottle, and cultured at constant temperature of 30° C. and 180 r/min for 11 h to prepare a seed solution of Saccharomyces cerevisiae CICC 32236. The concentration of the seed solution is adjusted so that the effective viable count is 1.5×10⁸ CFU/mL for use.

Candida utilis CGMCC 2.2878 is subjected to slant culture in the YPD agar medium. A single colony of 1-loop Saccharomyces cerevisiae is selected and plotted in the YPD agar medium, and cultured at 30° C. After slant culture, 1-loop Candida utilis CGMCC 2.2878 bevel is selected, inoculated into 100 mL of YPD liquid medium contained in a 250 mL triangular bottle, and cultured at constant temperature of 30° C. and 180 r/min for 11 h to prepare a seed solution of Candida utilis CGMCC 2.2878. The concentration of the seed solution is adjusted so that the effective viable count is 1.8×10⁸ CFU/mL for use.

After shake-flask culture, the obtained seed solution of Saccharomyces cerevisiae CICC 32236 and the seed solution of Candida utilis CGMCC 2.2878 are mixed in equal volume to obtain a compound bacterial solution, recorded as a compound microbial agent for use.

The following embodiments all use the Aspergillus niger microbial agent or compound microbial agent prepared here.

### Embodiment 1

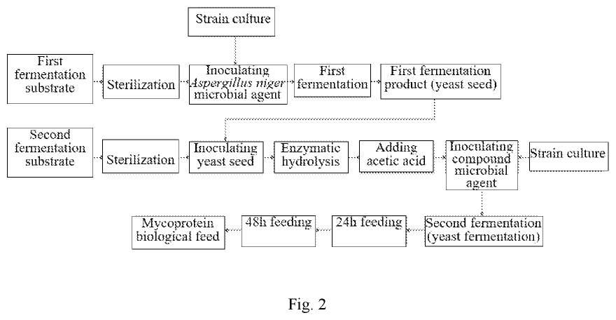

Dry corn husks are taken from Inner Mongolia Fufeng Biotechnology Co., Ltd. The water content of the dry corn husk is 9 wt. %. Brans are taken from Inner Mongolia Shenggu Brewing Food Co., Ltd. The soybean meal is taken from Inner Mongolia Shenggu Brewing Food Co., Ltd. The wheat middlings are taken from Inner Mongolia Shenggu Brewing Food Co., Ltd. Specific test step procedures are shown in FIG. 2, wherein the second fermentation substrate in FIG. 2 refers to the dry corn husks, molasses, ammonium sulfate, potassium dihydrogen phosphor, sodium chloride and magnesium sulfate in the second fermentation substrate:

### (1) Preparation of Fermentation Substrate

The first fermentation substrate (fermentation substrate 1) is composed of 86 parts of dry corn husks, 6 parts of brans, 4 parts of soybean meal and 4 parts of wheat middlings, wherein the dry corn husks are composed of coarse corn husks and fine corn husks, and the mass ratio of the coarse corn husks and the fine corn husks is 66:20. The specific mass after conversion is: 172 kg of dry corn husks, 12 kg of brans, 8 kg of soybean meal and 8 kg of wheat middlings, wherein the mass of the coarse corn husks is 132 kg, and the mass of the fine corn husks is 40 kg.

The second fermentation substrate (fermentation substrate 2) is composed of 19.5 parts of dry corn husks, 3 parts of molasses, 2 parts of ammonium sulfate, 0.2 part of potassium dihydrogen phosphate, 0.1 part of sodium chloride, 0.2 part of magnesium sulfate, 0.125 part of acetic acid and 10.5 parts of yeast seed, wherein the dry corn husks, molasses, ammonium sulfate, potassium dihydrogen phosphate, sodium chloride and magnesium sulfate are obtained after sterilized. Specific steps are provided in step (4). The dry corn husks are composed of coarse corn husks and fine corn husks, and the mass ratio of the coarse corn husks and the fine corn husks is 14.5:5. The specific mass after conversion is: 290 kg of coarse corn husks, 100 kg of fine corn husks, 60 kg of molasses, 40 kg of ammonium sulfate, 4 kg of potassium dihydrogen phosphate, 2 kg of sodium chloride, 4 kg of magnesium sulfate, 2.5 L of ethyl acid and 210 kg of yeast seed.

(2) Sterilization: The first fermentation substrate is put into a sterilization pot. After all the materials are added, steam of 120° C. and 0.2 MPa is introduced and stirred for 10 min. After pressure relief, tap water accounting for 80% of the mass of the first fermentation substrate is added, that is, 160 kg, and stirred for 10 min. Finally, steam of 120° C. and 0.2 MPa is introduced and stirred for 20 min to obtain the steaming material. The water content is controlled at 35 wt. %-40 wt. %, and the initial pH value is 7.0±0.2.

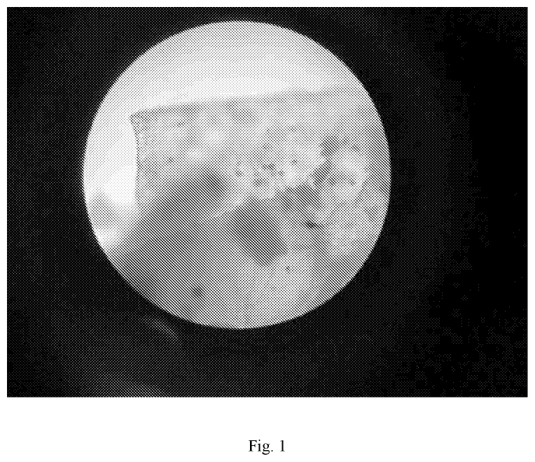

(3) After sterilization, the Aspergillus niger microbial agent is mixed directly with the steaming material. The material is transported with a screw conveyer, and the Aspergillus niger microbial agent is inoculated to ensure that the inoculation amount of the Aspergillus niger microbial agent is 5% of the mass of the first fermentation substrate. One truckload is firstly inoculated. About 50 kg of the Aspergillus niger microbial agent is inoculated for the first fermentation substrate, and then expanded inoculation is carried out to ensure that Aspergillus niger is uniformly inoculated to the fermentation substrate. The culture conditions of expanded inoculation are 30° C. and 48 h, and the substrate temperature after expanded inoculation is about 40° C. The “substrate temperature of about 40° C.” here refers to the temperature of the material after expanded culture in an Aspergillus niger tank. The inoculated substrate is sent to the Aspergillus niger tank by a trolley. The material layer is stacked obliquely. The material layer near an air inlet is 25 cm, and the material layer on the other side is 30 cm. The upper layer is shaved flat, and the first fermentation begins. The temperature of a fermentation chamber is controlled at 25° C.-30° C., the relative humidity is ≥95 wt. %, and the product temperature during intermittent ventilation is controlled at 30° C.-35° C. If the product temperature is higher than 35° C. and cannot be reduced to be less than 35° C. through continuous ventilation, cooling by turning or stirring is conducted. After the temperature is reduced to be less than 35° C., the first fermentation is continued for 5 h-10 h, and a large number of mycelia are observed and very robust, while a large number of spores are also present, as shown in FIG. 1. The operation is ended, and the yeast seed is obtained. The number of Aspergillus niger spores in the yeast seed reaches 6.52×10⁹ CFU/kg.

(4) Sterilization: The dry corn husks, molasses, ammonium sulfate, potassium dihydrogen phosphate, sodium chloride and magnesium sulfate in the second fermentation substrate are loaded into the sterilization pot, and then steam of 121° C. and 0.2 MPa is introduced and sterilized for 20 min; and 1400 kg of tap water is added to obtain the steaming material.

(5) After sterilization, the material in a steamer is sent to a yeast fermentation tank by a trolley, turned with the 210 kg of the above prepared yeast seed, and properly ventilated and cooled to ensure that the temperature of the corn husks is not less than 60° C. The tap water is added to adjust the water content to a slightly sticky shape, and the enzymatic hydrolysis reaction is conducted at 30° C. for 6 h. When liquid seeps at the bottom, the liquid is shoveled to the surface of the material layer in time.

(6) After the enzymatic hydrolysis, 2.5 L of acetic acid and compound microbial agent are added. The inoculation amount of the compound microbial agent is 5% of the mass of corn husks in the second fermentation substrate, which is converted to the specific volume as follows: 50 L of seed solution of Saccharomyces cerevisiae and 50 L of seed solution of Candida utilis are turned to start the second fermentation, which is recorded as yeast fermentation. The fermentation temperature is 30° C., and the fermentation time is 3 d. At the 24 h of fermentation, 1 wt. % ammonium sulfate of the total mass of the second fermentation substrate and 1% molasses of the total mass of the second fermentation substrate are added before the turning. At the 48 h of fermentation, 1% ammonium sulfate of the total mass of the second fermentation substrate and 1% molasses of the total mass of the second fermentation substrate are added before the turning. At the end of fermentation, the mycoprotein biological feed of corn husks, fermented by the step method, is obtained, which is recorded as a mycoprotein biological feed 1. The viable content of Candida utilis in the mycoprotein biological feed 1 is 1.9×10¹¹ CFU/kg, and the viable content of Saccharomyces cerevisiae is 1.4×10¹¹ CFU/kg.

### Embodiment 2

The steps are the same as those of embodiment 1, with the only difference in that: when step (3) is performed, the inoculation amount of the Aspergillus niger microbial agent is ensured to be 10% of the mass of the first fermentation substrate, and the mycoprotein biological feed of corn husks obtained at the end of fermentation is recorded as a mycoprotein biological feed 2.

### Embodiment 3

The steps are the same as those of embodiment 1, with the only difference in that: when step (1) is performed, the first fermentation substrate is replaced with a fermentation substrate 3, and other steps are unchanged. After fermentation, the mycoprotein biological feed of corn husks by multi-step fermentation is obtained, which is recorded as a mycoprotein biological feed 3.

Wherein the fermentation substrate 3 is composed of 82 parts of coarse corn husks, 10 parts of fine corn husks, 4 parts of brans, 2 parts of soybean meal and 2 parts of wheat middlings.

### Embodiment 4

The steps are the same as those of embodiment 1, with the only difference in that: when step (1) is performed, the first fermentation substrate is replaced with a fermentation substrate 4, and other steps are unchanged. After fermentation, the mycoprotein biological feed of corn husks by multi-step fermentation is obtained, which is recorded as a mycoprotein biological feed 4.

Wherein the fermentation substrate 4 is composed of 74 parts of coarse corn husks, 10 parts of fine corn husks, 6 parts of brans, 5 parts of soybean meal and 5 parts of wheat middlings.

### Embodiment 5

The steps are the same as those of embodiment 1, with the only difference in that: when step (6) is performed, the compound microbial agent is replaced with a compound microbial agent 2, and the other steps are unchanged. After fermentation, the obtained mycoprotein biological feed of corn husks is recorded as a mycoprotein biological feed 5.

Wherein the compound bacterial solution 2 is obtained by mixing the fermentation broth of Saccharomyces cerevisiae and the fermentation broth of Candida utilis in the preparation example according to a volume ratio of 7:3.

### Embodiment 6

The steps are the same as those of embodiment 1, with the only difference in that: when step (6) is performed, the compound bacterial solution is replaced with a compound bacterial solution 3, and other steps are unchanged. After fermentation, the obtained mycoprotein biological feed of corn husks is recorded as a mycoprotein biological feed 6.

Wherein the compound bacterial solution 3 is obtained by mixing the fermentation broth of Saccharomyces cerevisiae and the fermentation broth of Candida utilis in the preparation example according to a volume ratio of 3:7.

### Embodiment 7

The steps are the same as those of embodiment 1, with the only difference in that: when step (1) is performed, the second fermentation substrate is replaced with a fermentation substrate 5, and other steps are unchanged. After fermentation, the obtained mycoprotein biological feed of corn husks is recorded as a mycoprotein biological feed 7.

Wherein the fermentation substrate 5 is composed of 19.5 parts of corn husks, 10.5 parts of yeast seed prepared by embodiment 1, 5 parts of molasses, 4 parts of ammonium sulfate, 0.2 part of potassium dihydrogen phosphate, 0.1 part of sodium chloride, 0.2 part of magnesium sulfate and 0.125 part of acetic acid. Wherein the corn husks are composed of 14.5 parts of coarse corn husks and 5 parts of fine corn husks, which are converted to the specific mass as follows: 290 kg of coarse corn husks, 100 kg of fine corn husks, 210 kg of yeast seed, 100 kg of molasses, 80 kg of ammonium sulfate, 4 kg of potassium dihydrogen phosphate, 2 kg of sodium chloride, 4 kg of magnesium sulfate and 2.5 L of acetic acid.

### Embodiment 8

The steps are the same as those of embodiment 1, with the only difference in that: when step (1) is performed, the second fermentation substrate is replaced with a fermentation substrate 6, and other steps are unchanged. After fermentation, the obtained mycoprotein biological feed of corn husks is recorded as a mycoprotein biological feed 8.

Wherein the fermentation substrate 6 is composed of 19.5 parts of corn husks, 10.5 parts of yeast seed prepared by embodiment 1, 6 parts of molasses, 2 parts of ammonium sulfate, 0.1 part of potassium dihydrogen phosphate, 0.2 part of sodium chloride, 0.2 part of magnesium sulfate and 0.125 part of acetic acid, wherein the corn husks are composed of 14.5 parts of coarse corn husks and 5 parts of fine corn husks, which are converted to the specific mass as follows: 290 kg of coarse corn husks, 100 kg of fine corn husks, 210 kg of yeast seed, 120 kg of molasses, 40 kg of ammonium sulfate, 2 kg of potassium dihydrogen phosphate, 4 kg of sodium chloride, 4 kg of magnesium sulfate and 2.5 L of acetic acid.

### Application Example 1

Dairy cows in a lactation period are fed with the mycoprotein biological feeds of embodiments 1-8 to detect the influence of the mycoprotein biological feed of the present invention on milk yield of the dairy cows. Specific steps are as follows:

27 Holstein Black and White dairy cows are selected, randomly assigned and divided into 9 even groups. Each group has 3 dairy cows, respectively as follows: test group 0, test group 1, test group 2, test group 3, test group 4, test group 5, test group 6, test group 7 and test group 8. Wherein the dairy cows in the test group 0 are fed with wet corn husks with humidity of 35-45 wt. %, specifically including 14.5 parts of coarse corn husks and 5 parts of fine corn husks, which are converted to the specific mass: 290 kg of coarse corn husks and 100 kg of fine corn husks. The dairy cows in the test group 1 are fed with the mycoprotein biological feed 1. The dairy cows in the test group 2 are fed with the mycoprotein biological feed 2. The dairy cows in the test group 3 are fed with the mycoprotein biological feed 3. The dairy cows in the test group 4 are fed with the mycoprotein biological feed 4. The dairy cows in the test group 5 are fed with the mycoprotein biological feed 5. The dairy cows in the test group 6 are fed with the mycoprotein biological feed 6. The dairy cows in the test group 7 are fed with the mycoprotein biological feed 7. The dairy cows in the test group 8 are fed with the mycoprotein biological feed 8. The dairy cows in each test group are fed in a tethered mode, and fed by specially-assigned persons at 7 a.m. and 7 p.m. every day. The mode of free drinking and free eating is adopted, the dairy cows are milked at 4:30 and 15:30 every day, and the milk yield is recorded. A test period is 30 days.

Measurement of milk yield: At the beginning of feeding, the milk yield of each dairy cow in the test groups 0 to 8 is measured every day, and the average daily milk yield and total milk yield of each group of dairy cows are calculated.

Results show that at the end of the test, the daily milk yields of the test group 0, the test group 1, the test group 2, the test group 3, the test group 4, the test group 5, the test group 6, the test group 7 and the test group 8 are 24.8 kg±0.15 kg, 32.9 kg±0.27 kg, 27.8 kg±0.12 kg, 29.9 kg±0.45 kg, 30.8 kg±0.32 kg, 30.5 kg±0.22 kg, 30.2 kg±0.28 kg, 28.8 kg±0.19 kg and 32.5 kg±0.37 kg respectively; the total milk yields are 745 kg±2.35 kg, 987 kg±2.68 kg, 834 kg±6.54 kg, 897 kg±3.97 kg, 926 kg±1.23 kg, 915 kg±2.34 kg, 907 kg±5.62 kg, 864 kg±3.16 kg, and 975 kg±5.67 kg respectively; and the average daily milk yields and total milk yields of test groups 1 to 8 are significantly increased compared with those of the test group 0, indicating that the mycoprotein biological feeds of embodiments 1 to 8 can improve the milk yield of the dairy cows.

### Reference Example 1

The steps are the same as those of embodiment 1, with the only difference in that: when step (1) is performed, the second fermentation substrate is replaced with a fermentation substrate 7, and other steps are unchanged. After fermentation, the obtained mycoprotein biological feed of corn husks is recorded as a mycoprotein biological feed 9.

Wherein the fermentation substrate 7 is composed of 19.5 parts of dry corn husks, 10.5 parts of yeast seed prepared by embodiment 1, 3 parts of molasses, 6 parts of ammonium sulfate, 0.1 part of potassium dihydrogen phosphate, 0.2 part of sodium chloride, 0.2 part of magnesium sulfate and 0.125 part of acetic acid. Wherein the corn husks are composed of 14.5 parts of coarse corn husks and 5 parts of fine corn husks, which are converted to the specific mass as follows: 290 kg of coarse corn husks, 100 kg of fine corn husks, 210 kg of yeast seed, 60 kg of molasses, 120 kg of ammonium sulfate, 2 kg of potassium dihydrogen phosphate, 4 kg of sodium chloride, 4 kg of magnesium sulfate and 2.5 L of acetic acid.

### Reference Example 2

The steps are the same as those of embodiment 1, with the only difference in that: when step (1) is performed, the first fermentation substrate is replaced with a fermentation substrate 8. After fermentation, the obtained mycoprotein biological feed of corn husks is recorded as a mycoprotein biological feed 10.

Wherein the fermentation substrate 8 is composed of 19.5 parts of dry corn husks, 10.5 parts of yeast seed prepared by embodiment 1, 4 parts of molasses, 1 part of ammonium sulfate, 0.6 part of potassium dihydrogen phosphate, 0.3 part of sodium chloride, 0.2 part of magnesium sulfate and 0.125 part of acetic acid. Wherein the corn husks are composed of 14.5 parts of coarse corn husks and 5 parts of fine corn husks, which are converted to the specific mass as follows: 290 kg of coarse corn husks, 100 kg of fine corn husks, 210 kg of yeast seed, 80 kg of molasses, 20 kg of ammonium sulfate, 12 kg of potassium dihydrogen phosphate, 6 kg of sodium chloride, 4 kg of magnesium sulfate and 2.5 L of acetic acid.

### Reference Example 3

The steps are the same as those of embodiment 1, with the only difference in that: when step (1) is performed, the first fermentation substrate is replaced with a fermentation substrate 9, and other steps are unchanged. After fermentation, the mycoprotein biological feed of corn husks by multi-step fermentation is obtained, which is recorded as a mycoprotein biological feed 11.

Wherein the fermentation substrate 9 is composed of 19.5 parts of dry corn husks, 10.5 parts of yeast seed prepared by embodiment 1, 1 part of molasses, 5 parts of ammonium sulfate, 0.5 part of potassium dihydrogen phosphate, 0.3 part of sodium chloride, 0.2 part of magnesium sulfate and 0.125 part of acetic acid. Wherein the corn husks are composed of 14.5 parts of coarse corn husks and 5 parts of fine corn husks, which are converted to the specific mass as follows: 290 kg of coarse corn husks, 100 kg of fine corn husks, 210 kg of yeast seed, 20 kg of molasses, 100 kg of ammonium sulfate, 10 kg of potassium dihydrogen phosphate, 6 kg of sodium chloride, 4 kg of magnesium sulfate and 2.5 L of acetic acid.

### Reference Example 4

The steps are the same as those of embodiment 1, with the only difference in that: when step (1) is performed, the first fermentation substrate is replaced with a fermentation substrate 10, and other steps are unchanged. After fermentation, the mycoprotein biological feed of corn husks by multi-step fermentation is obtained, which is recorded as a mycoprotein biological feed 12.

Wherein the fermentation substrate 10 is composed of 19.5 parts of dry corn husks, 10.5 parts of yeast seed prepared by embodiment 1, 3 parts of molasses, 1 part of ammonium sulfate, 0.4 part of potassium dihydrogen phosphate, 0.3 part of sodium chloride, 0.1 part of magnesium sulfate and 0.125 part of acetic acid. Wherein the corn husks are composed of 14.5 parts of coarse corn husks and 5 parts of fine corn husks, which are converted to the specific mass as follows: 290 kg of coarse corn husks, 100 kg of fine corn husks, 210 kg of yeast seed, 60 kg of molasses, 20 kg of ammonium sulfate, 8 kg of potassium dihydrogen phosphate, 6 kg of sodium chloride, 2 kg of magnesium sulfate and 2.5 L of acetic acid.

### Test Example 1

Physical and chemical indexes and health indexes of the mycoprotein biological feeds prepared by embodiments 1-8 and reference examples 1-4 are determined. The determination methods of the physical and chemical indexes and the health indexes are as follows:

Physical and chemical indexes: The crude protein content is determined in accordance with GB/T6432-2018, the crude fiber content is determined in accordance with GB/T 6434-2006, the water content is determined in accordance with GB/T6435-2014, the crude ash content is determined in accordance with GB/T6438-1992, the crude fat content is determined in accordance with GB/T6433-2006, and the starch content is determined in accordance with GB/T 20194-2018.

Health indexes: The total arsenic content is determined in accordance with GB/T13079-2006; lead content is determined in accordance with GB/T13080-2004; mercury content is determined in accordance with GB/T13081-2006; cadmium content is determined in accordance with GB/T13082-1991; chromium content is determined in accordance with GB/T13088-2006; fluorine content is determined in accordance with GB/T13083-2002; the content of nitrite is determined in accordance with GB/T13085-2005; the content of aflatoxin B1 is determined in accordance with GB/T17480-1998; the content of ochratoxin A is determined in accordance with GB/T19539-2004; the content of zearalenone is determined in accordance with GB/T19540-2004; the content of vomitoxin is determined in accordance with GB/T30956-2014; the content of T-2 toxin is determined in accordance with GB/T28718-2012; the content of fumonisin is determined in accordance with NY/T1970-2010; the content of cyanide is determined in accordance with GB/T13084-2006; the content of free gossypol is determined in accordance with GB/T13086-1991; isothiocyanate content is determined in accordance with GB/T13087-1991; oxazolidinethione content is determined in accordance with GB/T13089-1991; hexachlorobenzene content is determined in accordance with GB/T34270-2017; the content of polychlorinated biphenyl is determined in accordance with GB/T 8381.8-2005; and salmonella is detected in accordance with GB/T 13091-2002. The specific process is the common knowledge and will not be described here.

A determination method of true protein is as follows:

### (1) Experimental Steps

About 1 g (accurate to 0.0001 g) of sample to be tested is accurately weighed, placed in a 200 ml beaker, added to 50 ml of water, and heated to boiling. Then, 20 ml of copper sulfate solution and 20 ml of 2.5% (m/m) sodium hydroxide aqueous solution are added, stirred fully with a glass rod, and left for more than 1 h. The solution is filtered with qualitative filter paper. Then, the precipitate is washed with hot water of 60-80° C. for 5 or 6 times. The filter paper is checked with 5 drops of barium chloride solution and 1 drop of hydrochloric acid solution, and the previous process of checking the filter paper with drops is repeated until no white barium sulfate precipitate is generated. The precipitate and the filter paper are dried in a 65° C. oven for 2 h, and then all transferred to a Kjeldahl flask for nitrogen determination by a semimicro Kjeldahl method.

### (2) Calculation Formula

$$True protein ( \% ) = \frac{( V 2 - V 1 ) \times C \times 0.014 \times 6.25}{m \times {V}^{′} \div V}$$

In the formula:
V2: the volume of a standard titration solution of hydrochloric acid consumed by a titration sample, in milliliters (mL);
V1: the volume of the standard titration solution of hydrochloric acid consumed by titration blank, in milliliters (mL);
C: the concentration of hydrochloric acid standard titration solution, in moles per liter (mol/L);
m: sample mass, in gram (g);
V: the total volume of a sample boiling solution, in milliliters (mL);
V: the volume of the boiling solution for distillation, in milliliters (mL);
6.25: an average coefficient of conversion of nitrogen to crude protein;

A detection method of Saccharomycetes is as follows:

1 g of mycoprotein biological feed is accurately weighed and mixed by vortex in 9 mL of sterile water for 15 min to produce a bacterial suspension with a Saccharomycetes concentration of 10⁻¹ cfu/mL. Then, 1 mL of bacterial suspension with a Saccharomycetes concentration of 10⁻¹ cfu/mL is absorbed, added to 9 mL of sterile water, fully oscillated and shaken, and diluted into a bacterial suspension with a Saccharomycetes concentration of 10⁻² cfu/mL. 10 μL of bacterial suspension with a Saccharomycetes concentration of 10⁻² cfu/mL is absorbed and dropped to a 0.0025 mm² counting area of a blood ball plate; then the same volume of methylene blue staining solution is dropped to make it evenly mixed; a cover glass is covered to ensure that no bubble is generated; the solution is stained for about 3-5 min; and the yeast activity is measured under a microscope within 5 min after staining and the viable count of yeast is calculated.

A calculation formula is:

$$yeast count / g = yeast count in 80 small cells / 80 \times 400 \times 1 {0}^{6} \times dilution ratio$$

Detection results are shown in Table 1, wherein % represents mass percent.

TABLE 1
Results of Detection Indexes of Mycoprotein Biological Feed

| | | | | | | | |
|---|---|---|---|---|---|---|---|
| | Embodi- | Embodi- | Embodi- | Embodi- | Embodi- | Embodi- | Embodi- |
| Index | ment 1 | ment 2 | ment 3 | ment 4 | ment 5 | ment 6 | ment 7 |

Physical and chemical indexes of mycoprotein biological feed

| | | | | | | | |
|---|---|---|---|---|---|---|---|
| Crude | 23.4 ± 0.1 | 21.5 ± 0.3 | 20.4 ± 0.6 | 22.5 ± 0.3 | 22.9 ± 0.6 | 22.7 ± 0.4 | 23.2 ± 0.3 |
| protein/% | | | | | | | |
| Crude | 16.1 ± 0.2 | 17.2 ± 0.5 | 17.8 ± 0.6 | 16.9 ± 0.4 | 16.8 ± 0.2 | 16.3 ± 0.3 | 16.6 ± 0.6 |
| fiber/% | | | | | | | |
| True | 16.9 ± 0.3 | 15.2 ± 0.4 | 14.9 ± 0.5 | 16.3 ± 0.3 | 17.2 ± 0.6 | 16.8 ± 0.4 | 19.3 ± 0.6 |
| protein/% | | | | | | | |
| Water/% | 52.6 ± 0.9 | 51.5 ± 0.8 | 55.3 ± 1.2 | 59.8 ± 1.5 | 54.3 ± 0.8 | 59.6 ± 0.2 | 62.6 ± 1.3 |
| Crude | 12.5 ± 0.4 | 13.4 ± 0.2 | 12.8 ± 0.3 | 12.2 ± 0.6 | 13.6 ± 0.6 | 14.2 ± 0.2 | 12.9 ± 0.8 |
| ash/% | | | | | | | |
| Crude | 5.6 ± 0.2 | 5.3 ± 0.3 | 5.8 ± 0.4 | 5.3 ± 0.1 | 5.4 ± 0.3 | 5.5 ± 0.2 | 5.6 ± 0.3 |
| fat/% | | | | | | | |
| Starch/% | 0.2 ± 0.02 | 0.2 ± 0.03 | 0.3 ± 0.04 | 0.2 ± 0.03 | 0.3 ± 0.02 | 0.4 ± 0.03 | 0.2 ± 0.02 |

Health indexes of mycoprotein biological feed- inorganic pollutants

| | | | | | | | |
|---|---|---|---|---|---|---|---|
| Total | Not | Not | Not | Not | Not | Not | Not |
| arsenic | detected | detected | detected | detected | detected | detected | detected |
| mg/kg | | | | | | | |
| Lead | Not | Not | Not | Not | Not | Not | Not |
| mg/kg | detected | detected | detected | detected | detected | detected | detected |
| Mercury | Not | Not | Not | Not | Not | Not | Not |
| mg/kg | detected | detected | detected | detected | detected | detected | detected |
| Cadmium | Not | Not | Not | Not | Not | Not | Not |
| mg/kg | detected | detected | detected | detected | detected | detected | detected |
| Chromium | Not | Not | Not | Not | Not | Not | Not |
| mg/kg | detected | detected | detected | detected | detected | detected | detected |
| Fluorine | Not | Not | Not | Not | Not | Not | Not |
| mg/kg | detected | detected | detected | detected | detected | detected | detected |
| Nitrite | Not | Not | Not | Not | Not | Not | Not |
| mg/kg | detected | detected | detected | detected | detected | detected | detected |

Health indexes of mycoprotein biological feed- mycotoxin

| | | | | | | | |
|---|---|---|---|---|---|---|---|
| Aflatoxin | Not | Not | Not | Not | Not | Not | Not |
| B1 μg/kg | detected | detected | detected | detected | detected | detected | detected |
| Ochratoxin | Not | Not | Not | Not | Not | Not | Not |
| A μg/kg | detected | detected | detected | detected | detected | detected | detected |
| Zearale | Not | Not | Not | Not | Not | Not | Not |
| none mg/kg | detected | detected | detected | detected | detected | detected | detected |
| Vomitoxin | 0.42 ± 0.02 | 0.53 ± 0.05 | 0.53 ± 0.10 | 0.48 ± 0.03 | 0.55 ± 0.08 | 0.59 ± 0.06 | 0.57 ± 0.05 |
| mg/kg | | | | | | | |
| T-2 toxin | Not | Not | Not | Not | Not | Not | Not |
| mg/kg | detected | detected | detected | detected | detected | detected | detected |
| Fumonisin | Not | Not | Not | Not | Not | Not | Not |
| (B1 + B2) | detected | detected | detected | detected | detected | detected | detected |
| mg/kg | | | | | | | |

Health indexes of mycoprotein biological feed- natural phytotoxin

| | | | | | | | |
|---|---|---|---|---|---|---|---|
| Cyanide | Not | Not | Not | Not | Not | Not | Not |
| mg/kg | detected | detected | detected | detected | detected | detected | detected |
| Free | Not | Not | Not | Not | Not | Not | Not |
| gossypol | detected | detected | detected | detected | detected | detected | detected |
| mg/kg | | | | | | | |
| Isothio- | Not | Not | Not | Not | Not | Not | Not |
| cyanate | detected | detected | detected | detected | detected | detected | detected |
| mg/kg | | | | | | | |
| Oxazolidine- | Not | Not | Not | Not | Not | Not | Not |
| thione | detected | detected | detected | detected | detected | detected | detected |
| mg/kg | | | | | | | |

Health indexes of mycoprotein biological feed- organic pollutants

| | | | | | | | |
|---|---|---|---|---|---|---|---|
| Hexachloro- | Not | Not | Not | Not | Not | Not | Not |
| benzene | detected | detected | detected | detected | detected | detected | detected |
| mg/kg | | | | | | | |
| Polychlo- | Not | Not | Not | Not | Not | Not | Not |
| rinated | detected | detected | detected | detected | detected | detected | detected |
| biphenyl | | | | | | | |
| μg/kg | | | | | | | |

Health indexes of mycoprotein biological feed- microorganism

| | | | | | | | |
|---|---|---|---|---|---|---|---|
| Salmonella | Not | Not | Not | Not | Not | Not | Not |
| CFU/g | detected | detected | detected | detected | detected | detected | detected |
| Aspergillus | 6.53 × 10⁶ | 6.62 × 10⁶ | 9.6 × 10⁶ | 5.6 × 10⁶ | 6.3 × 10⁷ | 5.9 × 10⁶ | 8.62 × 10⁵ |
| niger | | | | | | | |
| CFU/g | | | | | | | |
| Saccharomyces | 1.53 × 10⁸ | 1.36 × 10⁸ | 1.26 × 10⁹ | 1.68 × 10¹⁰ | 1.45 × 10⁸ | 1.86 × 10⁷ | 1.84 × 10⁸ |
| cerevisiae | | | | | | | |
| CFU/g | | | | | | | |
| Candida | 2.0 × 10⁸ | 1.9 × 10⁹ | 1.6 × 10⁹ | 1.4 × 10⁸ | 1.8 × 10⁸ | 2.3 × 10⁸ | 2.8 × 10⁹ |
| utilis | | | | | | | |
| CFU/g | | | | | | | |

| | Embodi- | Reference | Reference | Reference | Reference |
|---|---|---|---|---|---|
| Index | ment 8 | example 1 | example 2 | example 3 | example 4 |
| | Physical and chemical indexes of mycoprotein biological feed | | | | |
| Crude | 19.6 ± 0.4 | 16.3 ± 0.6 | 16.3 ± 0.2 | 17.0 ± 0.6 | 16.9 ± 0.1 |
| protein/% | | | | | |
| Crude | 18.5 ± 0.5 | 20.6 ± 0.6 | 20.3 ± 0.2 | 19.4 ± 0.5 | 18.1 ± 0.2 |
| fiber/% | | | | | |
| True | 12.4 ± 0.5 | 11.9 ± 0.3 | 9.2 ± 0.1 | 12.0 ± 0.8 | 10.7 ± 0.5 |
| protein/% | | | | | |
| Water/% | 61.3 ± 0.5 | 58.2 ± 0.5 | 56.3 ± 0.5 | 55.8 ± 0.9 | 53.4 ± 0.3 |
| Crude | 12.5 ± 0.1 | 13.6 ± 0.2 | 12.6 ± 0.5 | 13.8 ± 0.5 | 13.8 ± 0.1 |
| ash/% | | | | | |
| Crude | 5.8 ± 0.3 | 5.3 ± 0.3 | 5.2 ± 0.2 | 5.0 ± 0.4 | 5.1 ± 0.6 |
| fat/% | | | | | |
| Starch/% | 0.2 ± 0.05 | 0.2 ± 0.03 | 0.3 ± 0.02 | 0.4 ± 0.05 | 0.3 ± 0.08 |
| | Health indexes of mycoprotein biological feed- inorganic pollutants | | | | |
| Total | Not | Not | Not | Not | Not |
| arsenic | detected | detected | detected | detected | detected |
| mg/kg | | | | | |
| Lead | Not | Not | Not | Not | Not |
| mg/kg | detected | detected | detected | detected | detected |
| Mercury | Not | Not | Not | Not | Not |
| mg/kg | detected | detected | detected | detected | detected |
| Cadmium | Not | Not | Not | Not | Not |
| mg/kg | detected | detected | detected | detected | detected |
| Chromium | Not | Not | Not | Not | Not |
| mg/kg | detected | detected | detected | detected | detected |
| Fluorine | Not | Not | Not | Not | Not |
| mg/kg | detected | detected | detected | detected | detected |
| Nitrite | Not | Not | Not | Not | Not |
| mg/kg | detected | detected | detected | detected | detected |
| | Health indexes of mycoprotein biological feed- mycotoxin | | | | |
| Aflatoxin | Not | Not | Not | Not | Not |
| B1 μg/kg | detected | detected | detected | detected | detected |
| Ochratoxin | Not | Not | Not | Not | Not |
| A μg/kg | detected | detected | detected | detected | detected |
| Zearale | Not | Not | Not | Not | Not |
| none mg/kg | detected | detected | detected | detected | detected |
| Vomitoxin | 0.49 ± 0.06 | 0.65 ± 0.09 | 0.62 ± 0.03 | 0.63 ± 0.04 | 0.65 ± 0.01 |
| mg/kg | | | | | |
| T-2 toxin | Not | Not | Not | Not | Not |
| mg/kg | detected | detected | detected | detected | detected |
| Fumonisin | Not | Not | Not | Not | Not |
| (B1 + B2) | detected | detected | detected | detected | detected |
| mg/kg | | | | | |
| | Health indexes of mycoprotein biological feed- natural phytotoxin | | | | |
| Cyanide | Not | Not | Not | Not | Not |
| mg/kg | detected | detected | detected | detected | detected |
| Free | Not | Not | Not | Not | Not |
| gossypol | detected | detected | detected | detected | detected |
| mg/kg | | | | | |
| Isothio- | Not | Not | Not | Not | Not |
| cyanate | detected | detected | detected | detected | detected |
| mg/kg | | | | | |
| Oxazolidine- | Not | Not | Not | Not | Not |
| thione | detected | detected | detected | detected | detected |
| mg/kg | | | | | |
| | Health indexes of mycoprotein biological feed- organic pollutants | | | | |
| Hexachloro- | Not | Not | Not | Not | Not |
| benzene | detected | detected | detected | detected | detected |
| mg/kg | | | | | |
| Polychlo- | Not | Not | Not | Not | Not |
| rinated | detected | detected | detected | detected | detected |
| biphenyl | | | | | |
| μg/kg | | | | | |
| | Health indexes of mycoprotein biological feed- microorganism | | | | |
| Salmonella | Not | Not | Not | Not | Not |
| CFU/g | detected | detected | detected | detected | detected |
| Aspergillus | 7.6 × 10⁶ | 6.8 × 10⁷ | 9.6 × 10⁷ | 6.35 × 10⁷ | 6.68 × 10⁷ |
| niger | | | | | |
| CFU/g | | | | | |
| Saccharomyces | 1.67 × 10⁹ | 1.67 × 10⁷ | 1.02 × 10⁷ | 1.26 × 10⁷ | 1.16 × 10⁷ |
| cerevisiae | | | | | |
| CFU/g | | | | | |
| Candida | 1.6 × 10¹⁰ | 1.9 × 10⁷ | 1.5 × 10⁷ | 3.4 × 10⁷ | 2.8 × 10⁷ |
| utilis | | | | | |
| CFU/g | | | | | |

By comparing the data of embodiment 1 with that of the reference example 1, it can be seen that when the proportion of inorganic salt content is changed, the content of crude protein is lower than the standard value, the true protein content is significantly lower than that of embodiment 1, and the content of crude fiber is higher than the standard value. The reason from analysis may be improper distribution of the proportion of inorganic salt content, which inhibits the growth and reproduction of Saccharomycetes. Moreover, it can be seen from Table 1 that the number of Saccharomycetes in the reference example 1 is significantly lower than that in embodiments. By comparing the data of embodiment 2 with that of the reference example 2, it can be seen that when the inorganic nitrogen content is reduced, the content of crude protein is lower than the standard value, the true protein content is obviously lower than that of embodiment 2, and the content of crude fiber is higher than the standard value. The reason from analysis may be low content of available inorganic nitrogen source in the fermentation process of Saccharomycetes, which cannot meet the needs of own growth and reproduction. Moreover, it can be seen from Table 1 that the number of Saccharomycetes in the reference example 2 is significantly lower than that in embodiments 1-8.

By comparing the data of embodiment 3 with that of the reference example 3, it can be seen that when the content of inorganic nitrogen source is increased, the content of crude protein, the content of true protein and the content of crude fiber are all close to the standard values. The reason from analysis may be that increasing the content of inorganic nitrogen source can promote the growth and reproduction of Saccharomycetes, but the increase amplitude of nitrogen source content needs to be further improved.

By comparing the data of embodiment 6 with that of the reference example 2, it can be seen that when the concentration of molasses is increased, the content of crude protein and the content of true protein in the reference example 2 are significantly lower than those of embodiment 6 and the content of crude fiber is higher than the standard value, The reason from analysis may be high molasses concentration, which contains a large number of sugars, minerals, biotin, etc., thereby inhibiting the growth of Saccharomycetes. Thus, the content of crude protein and the content of true protein in the reference example 2 are low, and the number of Saccharomycetes is small.

By comparing the number of Saccharomycetes, it is found that the number of Saccharomycetes in embodiments 1-8 is higher than the number of Saccharomycetes in the reference example 1 to the reference example 4. It can be seen from Table 1 that through comparison of the above experimental data of embodiments 1-8 and the reference examples 1-4 and through comparison of the content of crude protein, the content of true protein and the content of crude fiber, the content of crude protein and the content of true protein in embodiments 1-8 are significantly higher than those of the reference examples 1-4, and meet the standard of protein feed; and the content of crude fiber is significantly lower than that of the reference examples, and meets the standard that the content of crude fiber in the protein feed is ≤18%. The content of inorganic pollutants in embodiments 1-8 of the present invention and the reference examples 1-4 is not detected; mycotoxin content meets the requirements of health index; and the content of natural toxins and organic pollutants is not detected. The probiotic content of the mycoprotein biological feed prepared by embodiments 1-8 of the present invention is significantly increased, that is, the number of Saccharomyces cerevisiae and Candida utilis is increased; the number of Aspergillus niger is decreased; and the feeding effect is obvious, and the milk yield of the dairy cows is increased.

In conclusion, the mycoprotein fermented feed of corn husks provided by the present invention can not only provide the high-quality protein, but also provide a variety of probiotics and enzymes, which has certain effects of reducing or eliminating the use of antibiotics and ensuring the green development of the breeding industry while ensuring the supply of the protein, and has wide application prospects.

Although the above embodiments provide detailed description of the present invention, the above embodiments are only part of the embodiments of the present invention, not all embodiments. Other embodiments can be obtained according to the embodiments without inventiveness, and these embodiments belong to the protection scope of the present invention.

1. A preparation method for a mycoprotein biological feed, comprising the following steps:
inoculating Aspergillus niger microbial agent on a first fermentation substrate for first fermentation, to obtain a yeast seed;
inoculating a compound microbial agent in a second fermentation substrate to perform second fermentation to obtain a mycoprotein biological feed;
wherein the first fermentation substrate comprises the following raw materials by mass parts: 84-92 parts of corn husks, 4-6 parts of brans, 2-4 parts of soybean meal and 2-5 parts of wheat middlings;
the second fermentation substrate comprises the following raw materials by mass parts: 19.5-30 parts of corn husks, 3-6 parts of molasses, 2-4 parts of ammonium sulfate, 0.1-0.2 part of potassium dihydrogen phosphate, 0.1-0.2 part of magnesium sulfate, 0.10-0.20 part of sodium chloride, 0.10-0.15 part of acetic acid and 10-35 parts of the yeast seed;
the compound microbial agent comprises Saccharomyces cerevisiae and Candida utilis.

2. The preparation method according to claim 1, wherein the inoculation amount of the Aspergillus niger is 5-10% of the mass of the first fermentation substrate;
the effective viable count of the Aspergillus niger is 1.0×10⁹-2.0×10⁹ CFU/mL.

3. The preparation method according to claim 1, wherein the Aspergillus niger comprises Aspergillus niger CJH-JXSFZh-B703 with a collection number of CGMCC No. 22439.

4. The preparation method according to claim 1, wherein the time of the first fermentation is 24-48 h, and the temperature is 30-35° C.

5. The preparation method according to claim 1, wherein the inoculation amount of the compound microbial agent is 5%-10% of mass of the corn husks in the second fermentation substrate;
the effective viable count of the Saccharomyces cerevisiae is 1.5×10⁸ CFU/mL;
the effective viable count of the Candida utilis is 1.8×10⁸ CFU/mL;
a ratio of the effective viable counts of the Saccharomyces cerevisiae and the Candida utilis in the compound microbial agent is 1.5-10.5:1.8-12.6.

6. The preparation method according to claim 1, wherein the Saccharomyces cerevisiae comprises Saccharomyces cerevisiae CICC 32236; and the Candida utilis comprises Candida utilis CGMCC 2.2878.

7. The preparation method according to claim 1, wherein the time of the second fermentation is 48-72 h and the temperature is 30-32° C.

8. A mycoprotein biological feed, which is prepared by the preparation method of claim 1.

9. The mycoprotein biological feed according to claim 8, wherein the effective viable count of the Saccharomyces cerevisiae in the mycoprotein biological feed is 1.4×10⁹ CFU/kg-1.4×10¹¹ CFU/kg, and the effective viable count of the Candida utilis is 1.9×10⁹ CFU/kg-1.9×10¹¹ CFU/kg.

10. An application of the mycoprotein biological feed according to claim 8 in preparation of an animal feed.

11. An application of the mycoprotein biological feed according to claim 8 in improvement of milk yield and milk quality of animals.
